(19) United States
(12) Patent Application Publication
(10) Pub. No.: US 2006/0166957 A1
(43) Pub. Date: Jul. 27, 2006

(54) Methods of treating obesity and related disorders using tellurium selenium compounds

(76) Inventors: Menachem Rubinstein, Rehovot (IL); Yossi Dagon, Rishon-LeZion (IL); Benjamin Sredni, Saba (IL); Michael Albeck, Ramat-Gan (IL)

Correspondence Address: Martin Moynihan;Prtsi Inc, PO Box 16446, Arlington, VA, 22215, US

(21) Appl. No.: 10/560,232

(22) PCT Filed: Jun. 13, 2004
(86) PCT No.: PCT/IL04/00506

§ 371 Date: Mar. 24, 2006

Related U.S. Application Data
(60) Provisional application No. 60/477,790, filed on Jun. 12, 2003.

(51) Int. Cl. A61K 31/33; A61K 31/185

(52) U.S. Cl. 514/183; 514/553

(57) ABSTRACT

The present invention relates to methods of using tellurium and selenium containing compounds, and particularly to the use of small organic molecules containing tellurium or selenium as exemplified by the compound ammonium-trichloro(dioxoethylene-O,O′)tellurate (known by the abbreviation AS101) for treating obesity and obesity related disorders or complications and for reducing of food intake.

### FIELD OF THE INVENTION

The present invention relates to methods for treating obesity and disorders related to obesity, and for reducing food intake, using tellurium- and selenium-containing compounds. In particular the present invention relates to use of small organic molecules comprising tellurium or selenium, including ammonium trichloro(dioxoethylene-O,O′)tellurate (known by the abbreviation AS101) in such methods.

### BACKGROUND OF THE INVENTION

The term obesity refers to an excess of adipose tissue relative to lean body mass. It is best viewed as any degree of excess adiposity that creates a health risk. The cutoff between normal and obese individuals can only be approximated, but the health risk imparted by obesity is probably a continuum with increasing adiposity. The most common value used to quantify obesity is the body mass index (BMI). BMI is defined as the ratio of a person's weight in kilograms and the square of their height expressed in meters. When a man's BMI is above 27.8, or a woman's exceeds 27.3; that person is considered overweight. The degree of obesity associated with a particular BMI ranges from mild obesity at a BMI near 27, moderate obesity at 30, severe obesity at 35, to very severe obesity at 40 or greater (Weighing the Options: Criteria for Evaluating Weight-Management Programs. Institute of Medicine, National Academy of Sciences. 1995; 50-51).

Obesity results from a greater consumption of energy than is used by the body. As this energy is stored, fat cells enlarge and increase in number, producing the characteristic pathology of obesity. The genetic makeup of human beings, which reflects a long evolutionary history of relative scarcity of foodstuffs, has run into an age of surfeit, and many people cannot readily adapt. Thus, the increased intake of food does not signal satiety, and there is a gradual increase in energy storage, particularly as intake of energy outpaces need as we grow older. Against this background of basic instincts unsuited to modern life in developed societies, it is possible to identity an increasing number of defects or etiologies that produce obesity. For most patients, however, it is not possible to connect obesity to a specific cause.

Obesity is associated with important psychological and medical morbidities, the latter including hypertension; dyslipidemia; type 2 diabetes; coronary heart disease; stroke; gallbladder disease; osteoarthritis; sleep apnea and respiratory problems; and endometrial, breast, prostate, and colon cancers. Higher body weights are also associated with increases in all-cause mortality. Obesity has reached epidemic status in the industrialized world. For example, about 97 million adults in the United States are overweight or obese. About 300,000 U.S. deaths a year are associated with obesity and overweight. The total direct and indirect costs attributed to overweight and obesity amounted to $117 billion in 2000. In 1999, an estimated 61 percent of U.S. adults were overweight, along with 13 percent of children and adolescents. Obesity among adults has doubled since 1980, while overweight among adolescents has tripled.

Treatment of obesity remains a problem. Except for exercise, diet and food restriction, currently there is no convincing pharmacological treatment for effective reduction of body weight. Plain diet usually fails due to poor compliance and when terminated, the patient returns to his pre-diet weight. One approved drug, Orlistat (Xenical), which inhibits lipase enzymes responsible for breaking down ingested fat Thus reduces fat adsorption through the gut, is only poorly effective. Moreover, some side effects with Orlistat include oily gas with discharge, urgent need to go to the bathroom, oily or fatty stools, an oily discharge, increased number of bowel movements, and inability to control bowel movements.

An alternative pharmacological approach is based on appetite suppressants. Several appetite suppressant medications have been proposed as treatment of obesity. Of these, only one appetite suppressant, sibutramine (Meridia) is approved for clinical use. In general, these medications are modestly effective, leading to an average weight loss of 5 to 22 pounds above that expected with non-drug obesity treatments. People respond differently to appetite suppressant medications, and some people experience more weight loss than others.

U.S. Pat. No. 6,403,641 discloses that co-administration of sibutramine hydrochloride monohydrate and Orlistat results in beneficial effects with respect to weight loss.

U.S. Pat. Nos. 6,624,161 and 6,656,934 disclose a particular class of benzoxazinone compounds, particularly 2-Oxy-benzoxazinone derivatives and 2-amino-benzoxazinone derivatives that has activity as lipase inhibitors, and are thus useful for the treatment of obesity and obesity-related diseases.

U.S. Pat. No. 6,476,059 relates to the use of polycyclic 2-aminothiazole systems and of their physiologically tolerated salts and physiologically functional derivatives for producing medicines for the prophylaxis or treatment of obesity.

Some obese patients using medication lose more than 10 percent of their starting body weight, an amount of weight loss that may reduce risk factors for obesity-related diseases, such as high blood pressure or diabetes. Maximum weight loss usually occurs within 6 months of starting medication treatment. Weight then tends to level off or increase during the remainder of treatment. Studies suggest that if a patient does not lose at least 4 pounds over 4 weeks on a particular medication, then that medication is unlikely to help the patient achieve significant weight loss. Few studies have looked at how safe or effective these medications are when taken for more than 1 year.

Some antidepressant medications have been studied as appetite suppressant medications. While these medications are FDA approved for the treatment of depression, their use in weight loss is an “off-label” use. Studies of these medications generally have found that patients lost modest amounts of weight for up to 6 months. However, most studies have found that patients who lost weight while taking antidepressant medications tended to regain weight while they were still on the drug treatment. Amphetamines and closely related compounds are not recommended for use in the treatment of obesity due to their potential for abuse and dependence.

The ob/ob mouse strain is very well studied as a model of human obesity. These spontaneously generated rodents do not express leptin, which is the adipocyte-generated signal of satiety (Y. Zhang et al., Nature 372, 425, 1994). As a result, ob/ob mice consume food continuously and can double or triple their weight, stored as fat, as compared with normal mice. In addition, ob/ob mice spontaneously develop insulin resistance, which resembles very much that of obese humans. So far, the only known treatment that can reverse the obesity and insulin resistance of ob/ob mice is exogenous leptin, administered by injection (M. A. Pelleymounter et al., Science 269, 540, 1995; J. L. Halaas et al., Science 269, 543, 1995; L. A. Campfield, et al, Science 269, 546, 1995). Of the currently approved anti obesity drugs, none has any significant effect on ob/ob mice. Clearly, any agent that can reverse the obese phenotype of ob/ob mice is a candidate for control of human obesity.

Albeck et al., U.S. Pat. No. 4,764,461, which is incorporated herein by reference, describes certain organic compounds of tellurium and selenium which are active in vitro and in vivo for the production of cytokines. These compounds are described as useful in the treatment of certain tumors, autoinmmune diseases, immune diseases and infectious diseases.

The nontoxic immunomodulator ammonium trichloro(dioxoethylene-O, O′)tellurate (AS101) has been shown to have beneficial effects in diverse preclinical and clinical studies. Most of its activities have been primarily attributed to the direct inhibition of the anti-inflammatory cytokine IL-10, followed by the simultaneous increase of specific cytokines. These include IL-1α, TNF-α, IFN-γ, IL-2, IL-12, and GM-CSF (Sredni, B. et al, 1987, Nature 330:173; Strassmann, G., et al, 1997, Cell. Immunol. 176:180; Kalechman, Y., et al, 1995, Blood 85:1555). These immunomodulatory properties were found to be crucial for the clinical activities of AS101, demonstrating the protective effects of AS101 in parasite- and viral-infected mouse models (Rosenblatt-Bin, H., et al, 1998, Cell. Immunol. 184:12), in autoimmune diseases (Kalechman, Y., et al, 1997, J. Immunol. 159:2658), and in a variety of tumor models in which AS101 had an antitumoral effect (Sredni, B., et al, 1996, J. Natl. Cancer Inst. 88:1276; Kalechman, Y., et al, 2000, Int. J. Cancer 86:281; Kalechman, Y., et al, 1996, J. Immunol. 156:1101). AS101 has also been shown to have protective properties against lethal and sublethal effects of irradiation and chemotherapy (Kalechman, Y., et al, 1990, J. Immunol. 145:1512; Kalechman, Y. et al, 1991, Cancer Res. 51:1499). These activities were also due to the increased production of proinflammatory cytokines and were associated with only minimal toxicity, thus enabling the use of the compound as an adjuvant to chemotherapy in phase II studies (Sredni, B. et al, 1995, J. Clin. Oncol. 13:2342).

### SUMMARY OF THE INVENTION

The present invention relates to novel uses of tellurium- and selenium-containing compounds for treatment of obesity and obesity related disorders or complications, including insulin resistance and type 2 diabetes. More particularly, the present invention provides methods of treating obesity and its associated complications by administering to an individual in need thereof a pharmaceutical composition comprising a therapeutically effective amount of a tellurium- or selenium-containing organic compound.

According to one aspect, the present invention provides a method of treating obesity comprising administering to an individual in need thereof a pharmaceutical composition comprising a therapeutically effective amount of a compound according to any one of formulae (I)-(VI): 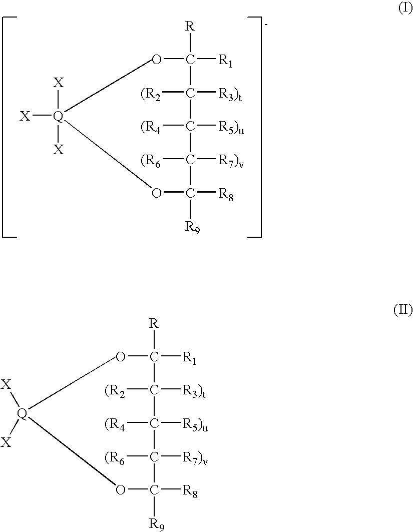 TeO₂ (III)
PhTeCl₃ (IV)
(C₆H₅)⁺P(TeCl₃(O₂C₂H₄))⁻ (V)

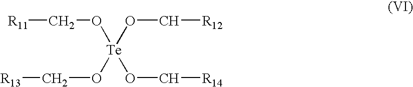

wherein Q is Te or Se; t is 1 or 0; u is 1 or 0; v is 1 or 0; R, R₁, R₂, R₃, R₄, R₅, R₆, R₇, R₈ and R₉ are the same or different and are independently selected from the group consisting of hydrogen, hydroxyalkyl of 1 to 5 carbons, hydroxyl, alkyl of from 1 to 5 carbon atoms, halogen, haloalkyl of 1 to 5 carbon atoms, carboxy, alkylcarbonylalkyl of 2 to 10 carbons, alkanoyloxy of 1 to 5 carbon atoms, carboxyalkyl of 1 to 5 carbon atoms, acyl, amido, cyano, amidoalkyl of 1 to 5 carbons, N-monoalkylamidoalkyl of 2 to 10 carbons, N,N-dialkylamidoalkyl of 4 to 10 carbons, cyanoalkyl of 1 to 5 carbons, alkoxy of 1 to 5 carbon atoms, alkoxyalkyl of 2 to 10 carbon atoms and —COR₁₀, wherein R₁₀ is alkyl of from 1 to 5 carbons; R₁₁, R₁₂, R₁₃ and R₁₄ are independently selected from the group consisting of hydrogen, hydroxyalkyl of 1-5 carbons atoms, hydroxyl and alkyl of 1-5 carbons atoms, X is halogen; and Y⁺ is a pharmaceutically acceptable cation.

According to one embodiment, Y⁺ is ammonium N According to another embodiment, X is chloro. It is to be understood that while the ammonium salt is currently preferred, other pharmaceutically acceptable salts are encompassed within the scope of the invention. According to one embodiment, the compounds with the five membered rings are preferred.

The present invention now discloses that surprisingly, compounds containing tellurium or selenium according to any one of formulae (I) to (VI) are effective in the treatment and/or prevention of obesity and obesity related disorders.

According to another aspect, the present invention provides a method of treating obesity related disorders comprising administering to an individual in need thereof a pharmaceutical composition comprising a therapeutically effective amount of a compound having any one of formulae (I) to (VI) as described herein above.

According to one embodiment, the obesity-related disorder is selected from the group consisting of insulin resistance, hypertension, dyslipidemia, hyperlipidemia, cardiovascular disease, stroke, gastrointestinal disease, gastrointestinal conditions, osteoarthritis, sleep apnea and respiratory problems, and eating disorders.

According to yet another aspect, the present invention provides a method of reducing food intake comprising administering to an individual in need thereof a pharmaceutical composition comprising a therapeutically effective amount of a compound having any one of formulae (I) to (VI) as described herein above.

According to a further aspect, the present invention provides a method of alleviating a disease or disorder by reduction of food intake comprising administering to an individual in need thereof a pharmaceutical composition comprising a therapeutically effective amount of a compound having any one of formula (I) to (VI) as described herein above.

According to one embodiment, the disease or disorder alleviated is selected from the group consisting of insulin resistance, hypertension, dyslipidemia, hyperlipidemia, cardiovascular disease, stroke, gastrointestinal disease, gastrointestinal conditions, osteoarthritis, sleep apnea and respiratory problems, and eating disorders.

The methods of the present invention are suitable for any mammal. According to one embodiment, the mammal is a human subject.

According to one embodiment, the compound to be used according to the methods of the present invention is selected from the group consisting of tellurium-based compounds having the formula (Ia):

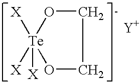

and formula (Ib):

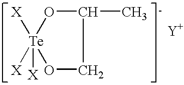

wherein X is halogen; Y⁺ is a pharmaceutically acceptable cation.

According to some embodiments of the present invention, the tellurium-based compounds are those of the formulae (Ia) and (Ib) wherein X is chloro. According to other embodiments the pharmaceutically acceptable cation is ammonium.

According to one currently preferred embodiment, the compound is ammonium trichloro(dioxoethylene-O,O′)tellurate, also known by the abbreviation AS101, having the formula:

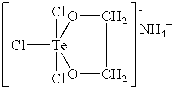

The present invention is based in part on the unexpected observation that administration of AS101 to ob/ob mice either by parenteral injection or orally in their drinking water, significantly reduced their food intake and body weight. In addition, AS101 treatment significantly reduced the blood glucose of the insulin-resistant ob/ob mice. Similarly, administration of AS101 to normal mice fed with a high fat diet significantly reduced their body weight. Thus, AS101, analogs thereof and pharmaceutical compositions comprising same are effective medicaments for reducing obesity and its associated complications.

According to the methods of the present invention, the compound of any one of formulae (I) to (VI) can be administered by any suitable route of administration, including but not limited to oral, parenteral, systemic, topical and transdermal routes of administration. The compounds may be administered orally in hard or soft gel liquid capsules, in solutions or suspensions, as capsules or tablets that may be prepared using conventional excipients, binders, disintegrating agents and the like. The parenteral route may be intramuscular, intravenous, intradermal using a sustained release carrier or subcutaneous. Formulations for extended release also fall within the scope of the invention. Such formulations include, for example, coated formulations and formulation comprising sustained release carriers.

The dosage of the compounds of the invention used for treatment according to the present invention may vary depending on the particular symptoms of the condition, disorder or disease, the stage in which the treatment is applied and the profile of the treated individual, for example the mammalian species and gender, age and weight.

The compounds of the present invention may be administered with or without pharmaceutical excipients, for example by administering the compound via the drinking water, or formulated to be administered as a pharmaceutical composition.

As used herein, a “pharmaceutical composition” refers to a preparation with one or more of the tellurium and selenium containing compounds described herein, or physiologically acceptable salts thereof, together with other chemicals components such as physiological acceptable diluents or carriers. The purpose of a pharmaceutical composition is to facilitate administration of a compound to an organism.

Pharmaceutical composition of the present invention may be manufactured by processes well known in the art, e.g. by means of conventional mixing, dissolving, granulating, grinding, pulverizing, dragee-making, levigating, emulsifying, encapsulating, entrapping or lyophilizing processes.

Pharmaceutical composition for use in accordance with the present invention thus may be formulated in conventional manner using one or more acceptable diluents or carriers comprising excipients and auxiliaries, which facilitate processing of the active compounds into preparations, which can be used pharmaceutically. Proper formulation is dependent on the route of administration chosen.

These and further embodiments will be better understood in conjugation with the description, figures and claims below.

### BRIEF DESCRIPTION OF THE DRAWING

FIG. 1 Shows the effect of orally administered AS101 on the weight of ob/ob mice in metabolic cages.

FIG. 2 Shows the effect of AS101 administration on daily food intake of ob/ob mice in metabolic cages.

FIG. 3 Shows the effect of AS101 on blood glucose of ob/ob mice.

FIG. 4 Shows the effect of AS101 on the fatty liver tissue of ob/ob mice.

FIG. 5 Shows the effect of AS101 on the weight of C56/BL mice that have been fed with a high fat diet.

FIG. 6 Shows the effect of AS101 on mouse 3T3 F442 cells induced to differentiate into adipocytes.

### DETAILED DESCRIPTION OF TE INENTION

The present invention provides novel use of small organic molecules comprising tellurium or selenium for the treatment of obesity in humans and other mammalian species.

According to certain embodiments the present invention provides methods of treating obesity and obesity related disorders or complications, and of reducing food intake, comprising administering to an individual in need thereof a pharmaceutical composition comprising a therapeutically effective amount of a selenium- or tellurium-containing compound.

According to specific embodiment, the compounds useful according to the present invention are those having any one of formulae (I)-(VI): 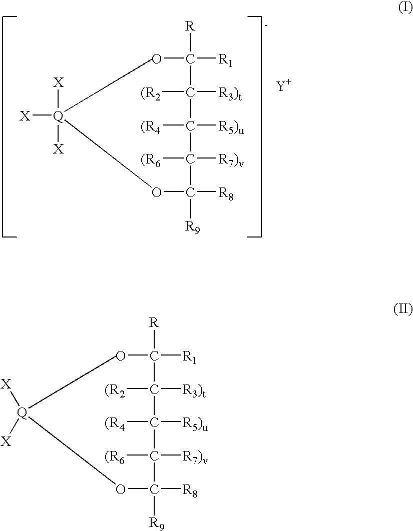 TeO₂ (III)
PhTeCl₃ (IV)
(C₆H₅)⁺P(TeCl₃(O₂C₂H₄))⁻ (V)

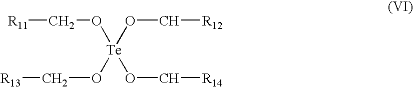

wherein Q is Te or Se; t is 1 or 0; u is 1 or 0; v is 1 or 0; R, R₁, R₂, R₃, R₄, R₅, R₆, R₇, R₈ and R₉ are the same or different and are independently selected from the group consisting of hydrogen, hydroxyalkyl of 1 to 5 carbons, hydroxyl, alkyl of from 1 to 5 carbon atoms, halogen, haloalkyl of 1 to 5 carbon atoms, carboxy, alkylcarbonylalkyl of 2 to 10 carbons, alkanoyloxy of 1 to 5 carbon atoms, carboxyalkyl of 1 to 5 carbon atoms, acyl, amido, cyano, amidoalkyl of 1 to 5 carbons, N-monoalkylamidoalkyl of 2 to 10 carbons, N,N-dialkylamidoalkyl of 4 to 10 carbons, cyanoalkyl of 1 to 5 carbons, alkoxy of 1 to 5 carbon atoms, alkoxyalkyl of 2 to 10 carbon atoms and —COR₁₀, wherein R₁₀ is alkyl of from 1 to 5 carbons; R₁₁, R₁₂, R₁₃ and R₁₄ are independently selected from the group consisting of hydrogen, hydroxyalkyl of 1-5 carbons atoms, hydroxyl and alkyl of 1-5 carbons atoms; X is halogen; and Y⁺ is a pharmaceutically acceptable cation. According to one embodiment, the compounds with the five membered rings are preferred.

As used herein and in the claims that follow, the term alkyl of 1 to 5 carbon atoms includes straight and branched chain alkyl groups such as methyl; ethyl; n-propyl; n-butyl, and the like; The alkyl may be unsubstituted or substituted by one or more substituents, i.e. substituents that do not interfere with the biological activity of the compounds. The term “substituted”, as used herein, means that any one or more hydrogen on the designated atom is replaced with a selection from the indicated group, provided that the designated atom's normal valency is not exceeded, and that the substitution results in a stable compound.

The term hydroxyalkyl of 1 to 5 carbon atoms includes hydroxymethyl; hydroxyethyl; hydroxy-n-butyl; the term haloalkyl of 1 to 5 carbon atoms includes chloromethyl; 2-iodoethyl; 4-bromo-n-butyl iodoethyl; 4-bromo-n-pentyl and the like; the term alkanoyloxy of 1 to 5 carbon atoms includes acetyl, propionyl, butanoyl and the like; the term carboxyalkyl includes carboxymethyl, carboxyethyl, ethylenecarboxy and the like; the term alkylcarbonylalkyl includes methanoylmethyl, ethanoylethyl and the like; the term amidoalkyl includes —CH₂CONH₂; —CH₂CH₂CONH₂; —CH₂CH₂CH₂CONH₂ and the like; the term cyanoalkyl includes —CH₂CN; —CH₂CH₂CN; —CH₂CH₂CH₂CN and the like; the term alkoxy of 1 to 5 carbon atoms includes methoxy, ethoxy, n-propoxy, n-pentoxy and the like; the terms halo and halogen are used to signify chloro, bromo, iodo and fluoro; the term acyl includes R₁₆CO wherein R₁₆ is H, or alkyl of 1 to 5 carbons such as methanoyl, ethanoyl and the like; the term aryl includes phenyl, alkylphenyl and naphthyl; the term N-monoalkylamidoalkyl includes —CH₂CH₂CONHCH₃, —CH₂CONHCH₂CH₃; the term N,N-dialkylamidoalkyl includes —CH₂CON(CH₃)₂; CH₂CH₂CON(CH₂ CH₃).

These compound and methods of their preparation are disclosed in U.S. Pat. Nos. 4,764,461, 5,271,925, 5,654,328, 6,552,089 to Albeck and Sredni, the teachings of which are incorporated herein in their entirety by reference. The methods of the present invention may be carried out using any member of an extended family of compounds containing tellurium or selenium, as well as their halides, or complexes of certain organic tellurium and selenium compounds with non-toxic complexing agents, the latter have increased water solubility for the preparation of aqueous pharmaceutical compositions as disclosed in the patents and other publications of Sredni, Albeck and coworkers.

According to one embodiment, the compounds which are based on tellurium are the presently preferred compounds of the invention. According to one currently preferred embodiment, the tellurium-based compounds are those of the formula (Ia):

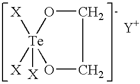

and formula (Ib):

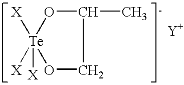

wherein X is halogen and Y⁺ is a pharmaceutically acceptable cation.

As used herein the term “halogen” refers to a member of the nonmetal halogen group located in group VIIA of the periodic table, particularly chloro, bromo, iodo and fluoro. According to some embodiments, preferred tellurium-based compounds are those wherein X is chloro. Y⁺ can be any pharmaceutically acceptable cation, including but not limited to the cation of the pharmaceutically acceptable salts described herein below. According to one preferred embodiments, the pharmaceutically acceptable cation is ammonium.

A particularly preferred embodiment of the present invention is ammonium trichloro(dioxoethylene-O,O′)tellurate (known by the abbreviation AS101), the structure of which is as follows:

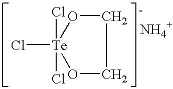

Other compounds which are based on tellurium and may be used in the practice of the invention include PhTeCl₃, TeO₂ and TeX₄(C₆H₅)₄P⁺(TeCl₃(O₂C₂H₄))⁻ (Z. Naturforsch, 36, 307-312 (1981)). Compounds of the following structure (formula IIa) are also included: 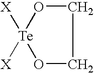
wherein X is a halogen, preferably chloro.

While the ammonium salt is illustrated, it is to be understood that other pharmaceutically acceptable salts are within the scope of the invention. Pharmaceutically acceptable salts are particularly suitable for medical application because of their greater solubility in water compared with the initial compounds on which they are based. As used herein, the term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

The compounds described above, specifically AS101 are used in the treatment of obesity. As used herein, the term “treatment” or “treating” is intended to include the administration of any one of the compounds of the invention to a subject for purposes which may include prophylaxis, amelioration, prevention or cure of obesity and obesity related disorders or complications. Such treatment need not necessarily completely ameliorate the disorder or other complications related to the specific disorder. Further, such treatment may be used in conjunction with other traditional treatments for treating obesity or a condition related to obesity, known to those of skill in the art.

The methods of the invention may be provided as a “preventive” treatment before a subject reaches the stages of sever obesity, so as to prevent the related disorder from developing.

The obesity related disorder is selected from the group consisting of, but not limited to insulin resistance, hyperglycaemia (type 2 diabetes), hypertension, dyslipidemia, hyperlipidemia, cardiovascular disease, stroke, gastrointestinal disease, gastrointestinal conditions, osteoarthritis, sleep apnea and respiratory problems, and eating disorders.

As described herein above, obesity is commonly defined by BMI of about 27 and over. However, it is to be understood that employing the compounds of the present invention for the treatment of over weight that does not fall under the definition of obesity is also encompassed within the scope of the present invention. The compounds of the present invention may be used for medical weight loss as well as for non-medical weight loss.

The “effective amount” of the compound which is necessary to achieve the desired biological effect, depends on a number of factors; for example the specific compound chosen the intended use, the mode of administration and the clinical condition of the patient. It is anticipated, however, that the dosages required to produce an anti-obesity effect are lower than those disclosed to be effective in any prior immunomodulatory uses of the material AS101.

For illustrative purposes, the use of AS101 in the treatment of obese mice is described. Two models of obesity in mice are provided. In the first model AS101 is administered in the drinking water of the genetically obese ob/ob mice. In the second model normal mice were rendered obese by a 3 month high-fat diet. AS101 was then administered by daily injections while the high-fat diet was continued. In both studies the AS101-treated group exhibited a significant loss of body weight and reduction of food intake. In case of the ob/ob mice a significant reduction of blood glucose was seen as well, demonstrating that AS101 reduces both the obesity and one of its major complications. Histological observations of the liver of ob/ob mice have demonstrated that AS101 significantly reduced the number of liver adipocytes as well as their size.

The results obtained according to the invention indicate that tellurium-containing compounds, specifically AS101 have utility in reducing food intake, and can be used as therapeutics to treat conditions that benefit from reduced food intake, such as obesity and its complications, for example insulin resistance and diabetes.

The present invention further relates to tellurate-containing agents, having an essentially homologous structure, which exhibit similar effects as AS101 on food intake and obesity. Example of such tellurate-containing agents include but not limited to agents obtained by substitution of aliphatic hydrogen residues by halogen radicals, by other functional groups, by extending the aliphatic group by additional methylene residues or by using double bonds instead of single bonds between carbon atoms. Such homologues are readily prepared by skilled organic chemists.

The methods of treatment of the present invention encompasses treatment by administration a therapeutically effective amount of a compound of any one of formulae (I) to (VI) and any other derivatives disclosed herein absent a diluent or carrier, as well as administration of a pharmaceutical composition comprising a therapeutically effective amount of a compound of any one of formulae (I) to (VI) and a pharmaceutically acceptable diluent or carrier.

The pharmaceutical compositions of the present invention can be formulated for administration by a variety of routes including oral, rectal, transdermal, subcutaneous, intravenous, intramuscular, intranasal, and topical. Such compositions are prepared in a manner well known in the pharmaceutical art and comprise as an active ingredient at least one compound according to any one of formulae (I) to (VI) and derivatives thereof as described herein above, further comprising an excipient or a carrier. During the preparation of the pharmaceutical compositions according to the present invention the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methylcellulose. The formulations can additionally include lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 0.01 to about 50 mg. The term “unit dosage forms” refers to physically discrete units suitable as unitary dosages for human subjects and other mammals, each unit containing a predetermined quantity of the active compound calculated to produce the desired therapeutic effect, in association with a suitable pharmaceutical excipient.

The active ingredient is effective over a wide dosage range and is generally administered in a therapeutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.01 to about 50 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings; such materials include a number of polymeric acids and mixtures of polymeric acids with materials such as shellac, cetyl alcohol, and cellulose acetate. Acid- and gastric fluid-resistant formulations are preferred. Suitable gastric fluid-resistant coatings comprise cellulose acetate phthalate, polyvinyl acetate phthalate, hydroxypropylmethylcellulose phthalate and anionic polymers of methacrylic acid and methyl methacrylate.

Suitable pharmaceutical compounds for oral administration may be in the form of separate units such as, for example, capsules, cachets, pastilles or tablets, each of which contains a defined amount of the selenium- or tellurium-containing compound according to the present invention; as powder or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. In general, the compositions are produced by uniform and homogeneous mixing of the active ingredient with a liquid and/or finely dispersed solid carrier, after which the product is shaped if necessary. Thus, for example, a tablet may be produced by compressing or shaping the powder or granules of the compound, where appropriate with one or more additional ingredients. Compressed tablets may be produced by tabletting the compound in free-flowing form, such as, for example, a powder or granules, where appropriate mixed with a binder, lubricant, inert diluent and/or one (or more) surface-active/dispersing agents in a suitable machine. Shaped tablets may be produced by shaping, in a suitable machine, the compound which is in powder form and has been moistened with an inert liquid diluent.

According to some embodiments, oral administration of the tellurium or selenium compounds according to the present invention may be given once daily, at a dose range of 0.01 mg/kg body weight to 7.5 mg/kg body weight, particularly from 0.01 mg/kg body weight to 0.75 mg/kg body weight. If desired, a dose regime based on alternate day therapy may be used.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Suitable pharmaceutical compositions for parenteral administration may be prepared by dissolving the compound in a suitable solvent such as an aqueous buffer and dimethyl sulfoxide or glycerol. The parenteral route may be intramuscular, intravenous, intradermal using a sustained release carrier or subcutaneous. The concentration of the compounds§in combination with a pharmaceutical carrier is not critical and is a matter of choice. Remingtons Practice of Pharmacy, 9th, 10th and 11th Ed. describe various pharmaceutical carriers and is incorporated herein by reference.

According to certain embodiments, parenteral administration may be at a dose range of from about 0.01 mg/kg body weight per day to 1.0 mg/kg body weight per day. Alternatively, the same dosage may be given every other day.

Pharmaceutical compositions for parenteral administration may be formulated for immediate as well as sustained release. Sustained release formulations may include certain carriers which prolong the duration of the release of the active ingredient.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252 incorporated herein by reference as if fully set forth. Such patches may be constructed for continuous, pulsatile, or on demand delivery of pharmaceutical agents. Such patches suitably contain the active ingredient in an aqueous solution which is buffered where appropriate, dissolved and/or dispersed in an adhesive or dispersed in a polymer.

Suitable pharmaceutical compositions for topical use on the skin are preferably in the form of an ointment, cream, lotion, paste, spray, aerosol or oil. Examples of suitable vehicles include petrolatum, Aquaphor, Neobase, propylene glycol, glycerin and the like. These base materials are described in Remington's Pharmaceutical Sciences 17th Ed. Mack Publishing (1985), pp. 1301-1306 which is incorporated herein by reference. Combinations of two or more of theses vehicle can also be used. According to some embodiment, the compounds of the present invention are administered topically at a dose range of from about 0.01 mg/kg body weight per day to 2.5 mg/kg body weight per day.

In another aspect, the invention relates to a method for reduction of food intake or for treatment of a disease or disorder that can be alleviated by reduction of food intake which comprises administering to an individual in need thereof an effective amount of a compound having any one of formulae (I) to (VI). Any disease or disorder known today or to be discovered in the future that can be alleviated by reduction of food intake such as, but not limited to, obesity, hypertension, dyslipidemia, hyperlipidemia, cardiovascular risk, stroke, gastrointestinal disease, gastrointestinal conditions, eating disorder, insulin-resistance, and diabetes mellitus, is envisaged for treatment with selenium- and tellurium-containing compounds according to the present invention. According to one currently preferred embodiment, the compound used in the reduction of food intake is a tellurium-containing compound. According to another currently preferred embodiment, the compound is AS101.

The invention will now be illustrated by the following non-limiting Examples.

### EXAMPLES

### Example 1

### AS101 Reduces the Food Intake, Body Weight and Blood Glucose of ob/ob Mice

Female ob/ob mice (8 weeks old) were placed in metabolic cages and were given free drinking water and chow. After 48 h, one group of 3 mice (the Control group) continued to receive water and chow. A second group of 3 mice received drinking water containing AS 101 (7 mg/L) in addition to the standard chow. The experiment was continued for 18 days and body weights were measured daily. On the average the mice consumed about 1.5 ml of water per 24 h, corresponding to 10 microgram AS101 per mouse per day. This value corresponds to 0.25 mg/kg body weight per 24 h. Preliminary toxicity studies in mice have been previously shown (U.S. Pat. No. 4,764,461) an LD₅₀ of 300 μg/25 g of body weight in 6-week-old mice (12 mg/kg body weight); the concentration shown in the present invention to be effective is significantly lower, thus may be considered as a non-toxic concentration.

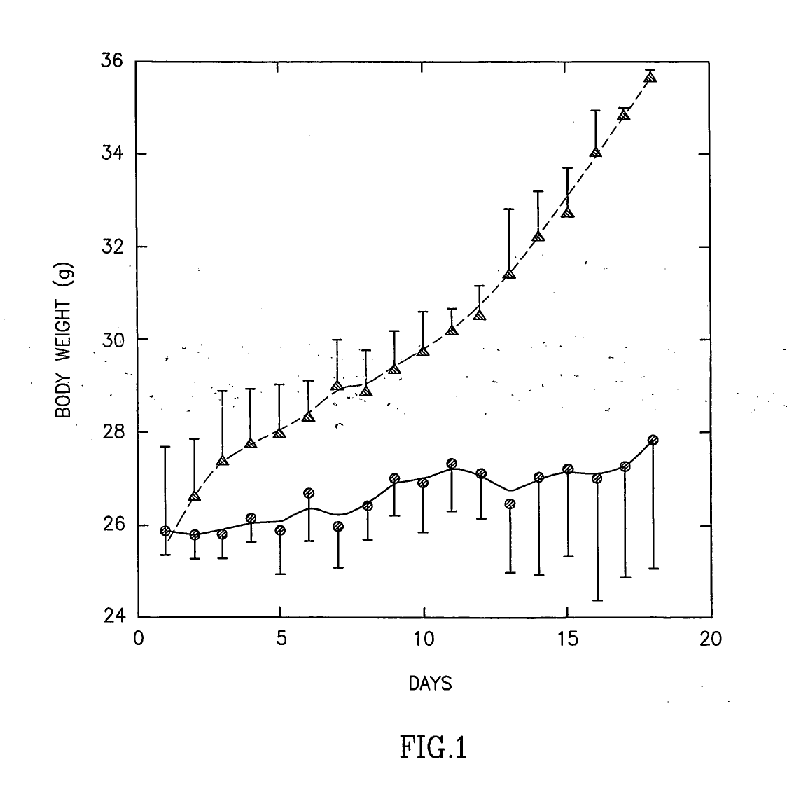

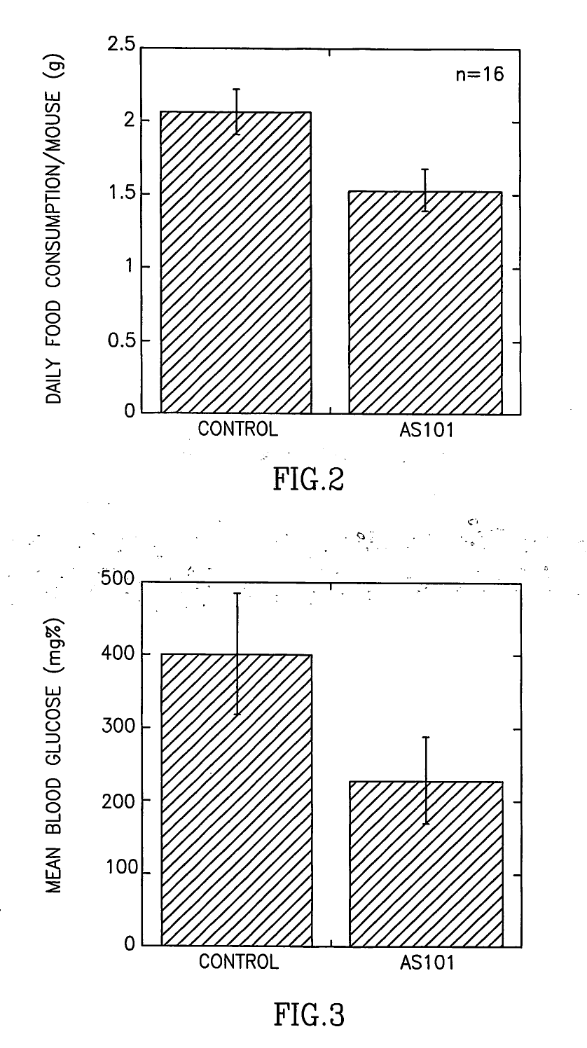

Measurement of the body weight revealed that the Control group gained weight continuously, whereas the AS101 group did not gain weight significantly (FIG. 1). Measurement of daily food consumption revealed a statistically significant reduction of food intake in the AS101 group, in line with the reduction in weight gain. On the average, food consumption was reduced by 26±7% (p<0.02, n=16, FIG. 2).

On the last day of treatment, tail blood was withdrawn and blood glucose was determined in three control mice and three mice of the AS101 group. A statistically significant (p<0.05) reduction of blood glucose was observed (FIG. 3) in the AS101 group.

### Example 2

### AS101 Reduces Cellularity and Adipocyte Size in the Adipose Tissue of ob/ob Mice

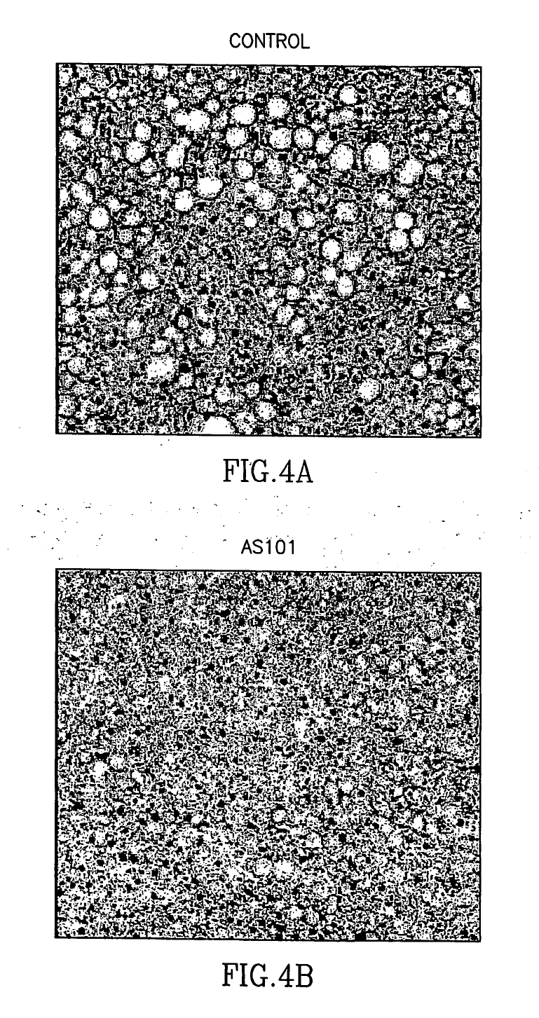

Following 18 days treatment of the ob/ob mice with AS101 as described in Example 1, the animals were sacrificed. Samples of liver tissue were taken, fixed with formaldehyde and tissue slices were examined microscopically (FIG. 4). An extensive reduction in the number of adipocytes and in their size was obtained in liver tissues of AS101-treated mice (FIG. 4B) as compared with control mice (FIG. 4 A). This result demonstrates the efficacy of AS101 treatment in reduction of the adipose tissue.

### Example 3

### AS101 Reduces the Body Weight of Diet-Induced Obese C57BL/6 Mice

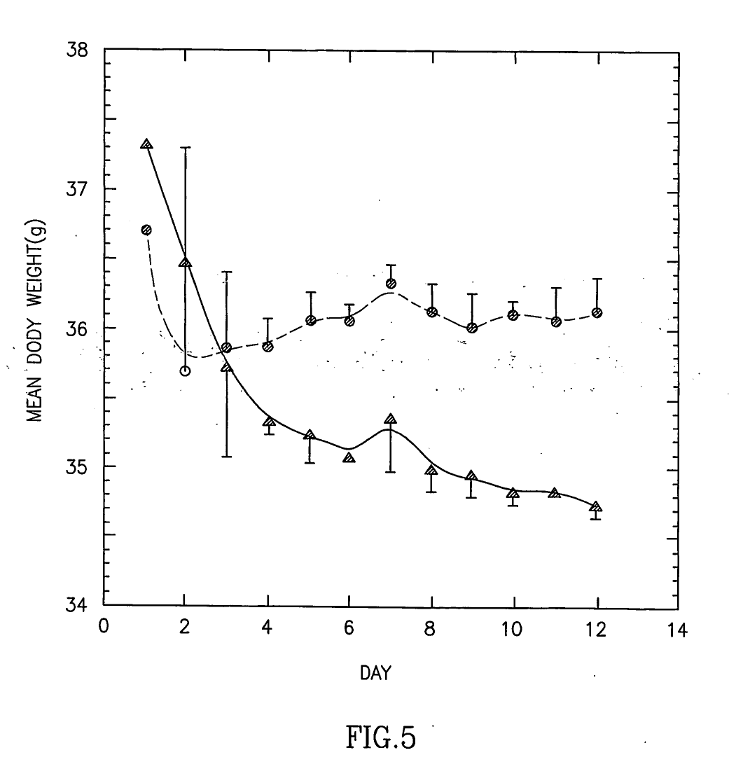

Since the obesity of ob/ob mice is of genetic origin, whereas obesity among human populations is largely due to excess eating over energy expenditure, the effect of AS101 on body weight was studied in diet-induced obese mice. Female mice were fed for 10 weeks with a high fat diet, resulting in moderate obesity (average weight 37 g). Mice were placed in metabolic cages and injected daily with either saline (Control group, 3 mice) or AS 101 (0.5 mg/kg body weight in saline, AS101 group, 3 mice). High fat diet was continued and the body weight was determined daily. As can be seen in FIG. 5, after an initial drop in weight due to stress in the metabolic cage the weight of the control group remained stable, whereas the weight of AS101-treated mice was reduced significantly and continued to drop through the entire study. Therefore, AS101 is an effective medicament for the treatment of diet-induced obesity.

### Example 4

### AS101 Inhibits the Differentiation of Pre-Adipocytes into Adipocytes

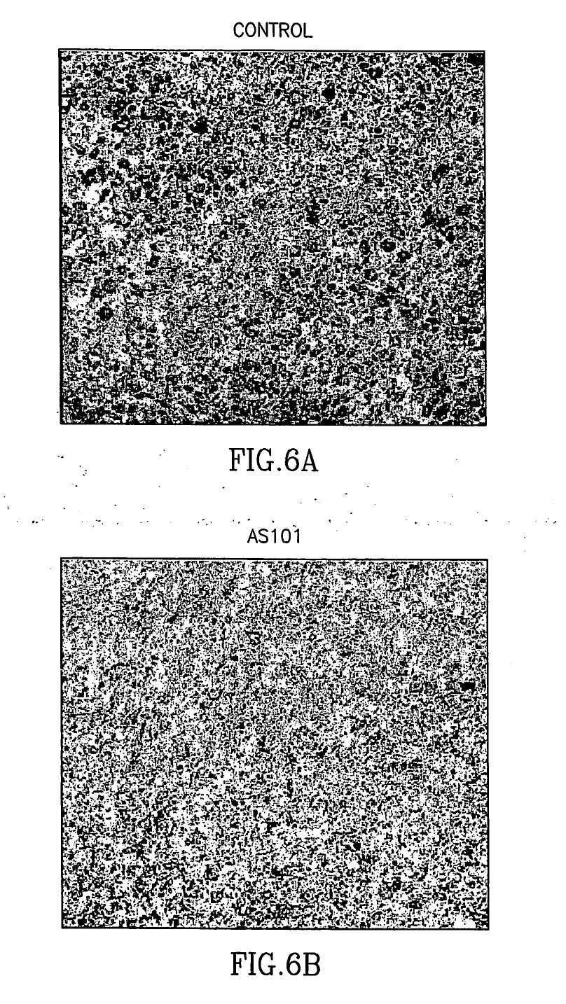

To gain insight on the mechanism by which AS101 affects the adipose tissue, its effect was studied on an in-vitro model of differentiation of pre-adipocytes into mature adipocytes. It should be clarified here that adipocytes are fully differentiated cells. As such, they do not proliferate. Besides increase in cell volume, the only way to gain weight in vivo is by generating new adipocytes from pre-adipocytes by differentiation. Swiss 3T3-F442A murine pre-adipocytes were grown in DMEM (GIBCO, USA) with 10% calf serum. To induce differentiation into mature adipocytes, confluent cell cultures were maintained in DMEM supplemented with 10% fetal bovine serum (FBS) for six days, either in the presence or in the absence of AS101 (0.2 micrograms/ml). The cultures were then stained with oil red, which specifically stains the fat droplets within the cells. As can be seen, AS101 inhibited the differentiation of 3T3-F442A pre-adipocytes into mature adipocytes as determined by the presence (FIG. 6A) or absence (FIG. 6B) of the fat droplets (visible in the figure as dark spots). Hence AS101 acts as an inhibitor of adipocyte generation.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A method of treating obesity comprising administering to an individual in need thereof a pharmaceutical composition comprising a therapeutically effective amount of a compound having any one of formulae (I) (VI): 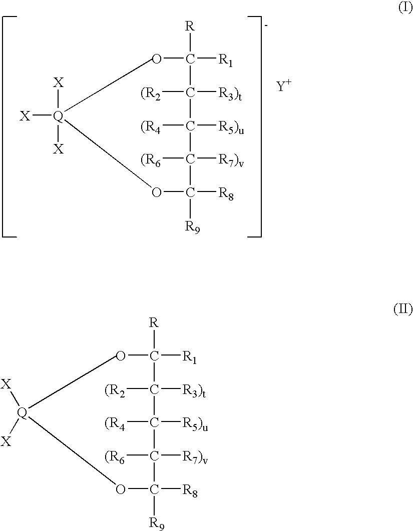
TeO₂ (III)
PhTeCl₃ (IV)
(C₆H₅)⁺P(TeCl₃(O₂C₂H₄))⁻ (V) 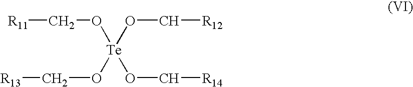
wherein Q is Te or Se; t is 1 or 0; u is 1 or 0; v is 1 or 0; R, R₁, R₂, R₃, R₄, R₅, R₆, R₇, R₈ and R₉ are the same or different and are independently selected from the group consisting of hydrogen, hydroxyalkyl of 1 to 5 carbons, hydroxyl, alkyl of from 1 to 5 carbon atoms, halogen, haloalkyl of 1 to 5 carbon atoms, carboxy, alkylcarbonylalkyl of 2 to 10 carbons, alkanoyloxy of 1 to 5 carbon atoms, carboxyalkyl of 1 to 5 carbon atoms, acyl, amido, cyano, amidoalkyl of 1 to 5 carbons, N-monoalkylamidoalkyl of 2 to 10 carbons, N,N-dialkylamidoalkyl of 4 to 10 carbons, cyanoalkyl of 1 to 5 carbons, alkoxy of 1 to 5 carbon atoms, alkoxyalkyl of 2 to 10 carbon atoms and —COR₁₀, wherein R₁₀ is alkyl of from 1 to 5 carbons; R₁₁, R₁₂, R₁₃ and R₁₄ are independently selected from the group consisting of hydrogen, hydroxyalkyl of 1-5 carbons atoms, hydroxyl and alkyl of 1-5 carbons atoms; X is halogen; Y⁺ is a pharmaceutically acceptable cation.

2. The method of claim 1, wherein Q is Te.

3. The method of claim 2, wherein Y⁺ is NH₄⁺.

4. The method of claim 2, wherein the compound has the formula: 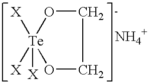
wherein X is halogen.

5. The method of claim 4, wherein the compound is ammonium trichloro(dioxoethylene-O,O′)tellurate (AS101).

6. The method of claim 1 wherein the individual is a human subject.

7. The method of claim 1 wherein the individual is a non-human mammal.

8. The method of claim 1 wherein the pharmaceutical composition is administered orally, parenterally, transdermally, topically or by contacting mucous membranes.

9. The method of claim 8 wherein the pharmaceutical composition is administered orally in a unit dosage form selected from solutions, suspensions, capsules and tablets.

10. The method of claim 8 wherein the pharmaceutical composition is administered via a parenteral route selected from intramuscular, intravenous, intradermal and subcutaneous.

11. The method of claim 8 wherein the pharmaceutical composition is suitable for sustained or controlled release.

12. A method of treating obesity related disorders comprising administering to an individual in need thereof a pharmaceutical composition comprising a therapeutically effective amount of a compound having any one of formulae (I)-(VI): 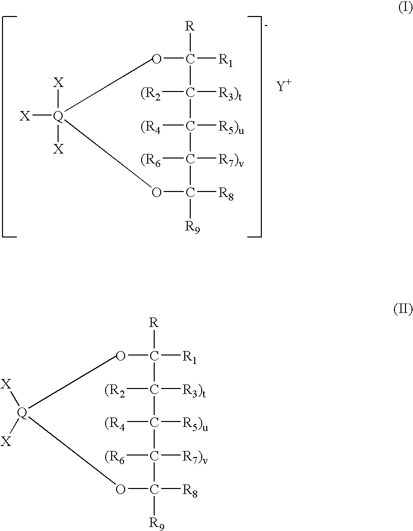
TeO₂ (III)
PhTeCl₃ (IV)
(C₆H₅)⁺P(TeCl₃(O₂C₂H₄))⁻ (V) 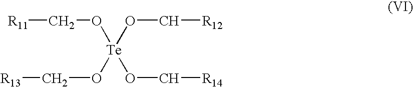
wherein Q is Te or Se; t is 1 or 0; u is 1 or 0; v is 1 or 0; R, R₁, R₂, R₃, R₄, R₅, R₆, R₇, R₈ and R₉ are the same or different and are independently selected from the group consisting of hydrogen, hydroxyalkyl of 1 to 5 carbons, hydroxyl, alkyl of from 1 to 5 carbon atoms, halogen, haloalkyl of 1 to 5 carbon atoms, carboxy, alkylcarbonylalkyl of 2 to 10 carbons, alkanoyloxy of 1 to 5 carbon atoms, carboxyalkyl of 1 to 5 carbon atoms, acyl, amido, cyano, amidoalkyl of 1 to 5 carbons, N-monoalkylamidoalkyl of 2 to 10 carbons, N,N-dialkylamidoalkyl of 4 to 10 carbons, cyanoalkyl of 1 to 5 carbons, alkoxy of 1 to 5 carbon atoms, alkoxyalkyl of 2 to 10 carbon atoms and —COR₁₀, wherein R₁₀ is alkyl of from 1 to 5 carbons; R₁₁, R₁₂, R₁₃ and R₁₄ are independently selected from the group consisting of hydrogen, hydroxyalkyl of 1-5 carbons atoms, hydroxyl and alkyl of 1-5 carbons atoms; X is halogen; and Y⁺ is a pharmaceutically acceptable cation.

13. The method of claim 12, wherein Q is Te.

14. The method of claim 13, wherein Y⁺ is NH₄⁺.

15. The method of claim 14, wherein the compound has the formula: 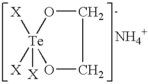
wherein X is halogen.

16. The method of claim 15, wherein the compound is ammonium trichloro(dioxoethylene-O,O′)tellurate (AS101).

17. The method of claim 12 wherein the obesity related disorder is selected from insulin resistance; hypertension; dyslipidemia; hyperlipidemia, cardiovascular disease; stroke; gastrointestinal disease; gastrointestinal conditions; osteoarthritis; sleep apnea and respiratory problems; and eating disorders.

18. The method of claim 12 wherein the individual is a human subject.

19. The method of claim 12 wherein the individual is a non-human mammal.

20. The method of claim 12 wherein the pharmaceutical composition is administered orally, parenterally, transdermally, topically or by contacting mucous membranes.

21. The method of claim 20 wherein the pharmaceutical composition is administered orally in unit dosage forms selected from solutions, suspensions, capsules and tablets.

22. The method of claim 20 wherein the pharmaceutical composition is administered via a parenteral route selected from intramuscular, intravenous, intradermal and subcutaneous.

23. The method of claim 20 wherein the pharmaceutical composition is suitable for sustained or controlled release.

24. A method of reducing food intake comprising administering to an individual in need thereof a pharmaceutical composition comprising a therapeutically effective amount of a compound having any one of formulae (I)-(VI): 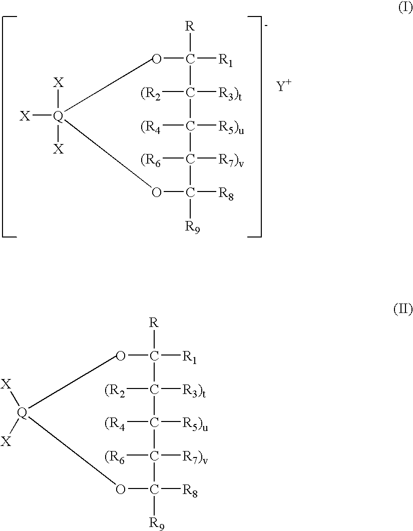
TeO₂ (III)
PhTeCl₃ (IV)
(C₆H₅)⁺P(TeCl₃(O₂C₂H₄))⁻ (V) 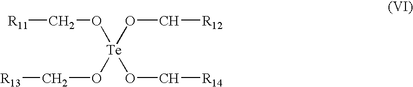
wherein Q is Te or Se; t is 1 or 0; u is 1 or 0; v is 1 or 0; R, R₁, R₂, R₃, R₄, R₅, R₆, R₇, R₈ and R₉ are the same or different and are independently selected from the group consisting of hydrogen, hydroxyalkyl of 1 to 5 carbons, hydroxyl, alkyl of from 1 to 5 carbon atoms, halogen, haloalkyl of 1 to 5 carbon atoms, carboxy, alkylcarbonylalkyl of 2 to 10 carbons, alkanoyloxy of 1 to 5 carbon atoms, carboxyalkyl of 1 to 5 carbon atoms, acyl, amido, cyano, amidoalkyl of 1 to 5 carbons, N-monoalkylamidoalkyl of 2 to 10 carbons, N,N-dialkylamidoalkyl of 4 to 10 carbons, cyanoalkyl of 1 to 5 carbons, alkoxy of 1 to 5 carbon atoms, alkoxyalkyl of 2 to 10 carbon atoms and —COR₁₀, wherein R₁₀ is alkyl of from 1 to 5 carbons; R₁₁, R₁₂, R₁₃ and R₁₄ are independently selected from the group consisting of hydrogen, hydroxyalkyl of 1-5 carbons atoms, hydroxyl and alkyl of 1-5 carbons atoms; X is halogen; and Y⁺ is a pharmaceutically acceptable cation.

25. The method of claim 24, wherein Q is Te.

26. The method of claim 25, wherein Y⁺ is NH₄⁺.

27. The method of claim 26, wherein the compound has the formula: 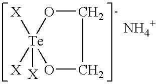
wherein X is halogen.

28. The method of claim 27, wherein the compound is ammonium trichloro(dioxoethylene-O,O′)tellurate (AS101).

29. The method of claim 24 wherein the individual is a human subject.

30. The method of claim 24 wherein the individual is a non-human mammal.

31. The method of claim 24 wherein the pharmaceutical composition is administered orally, parenterally, transdermally, topically or by contacting mucous membranes.

32. The method of claim 31 wherein the pharmaceutical composition is administered orally in unit dosage forms selected from solutions, suspensions, capsules and tablets.

33. The method of claim 31 wherein the pharmaceutical composition is administered via a parenteral route selected from intramuscular, intravenous, intradermal and subcutaneous.

34. The method of claim 31 wherein the pharmaceutical composition is suitable for sustained or controlled release.

35. A method of alleviating a disease or disorder by reduction of food intake comprising administering to an individual in need thereof a pharmaceutical composition comprising a therapeutically effective amount of a compound having any one of formulae (I)-(VI): 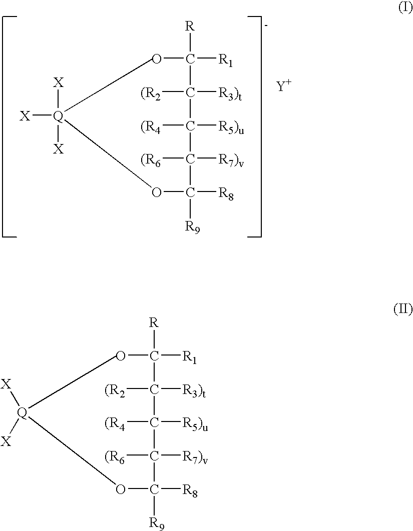
TeO₂ (III)
PhTeCl₃ (IV)
(C₆H₅)⁺P(TeCl₃(O₂C₂H₄))⁻ (V) 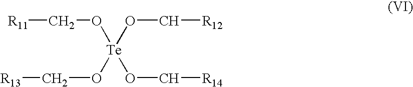
wherein Q is Te or Se; t is 1 or 0; u is 1 or 0; v is 1 or 0; R, R₁, R₂, R₃, R₄, R₅, R₆, R₇, R₈ and R₉ are the same or different and are independently selected from the group consisting of hydrogen, hydroxyalkyl of 1 to 5 carbons, hydroxyl, alkyl of from 1 to 5 carbon atoms, halogen, haloalkyl of 1 to 5 carbon atoms, carboxy, alkylcarbonylalkyl of 2 to 10 carbons, alkanoyloxy of 1 to 5 carbon atoms, carboxyalkyl of 1 to 5 carbon atoms, acyl, amido, cyano, amidoalkyl of 1 to 5 carbons, N-monoalkylamidoalkyl of 2 to 10 carbons, N,N-dialkylamidoalkyl of 4 to 10 carbons, cyanoalkyl of 1 to 5 carbons, alkoxy of 1 to 5 carbon atoms, alkoxyalkyl of 2 to 10 carbon atoms and —COR₁₀, wherein R₁₀ is alkyl of from 1 to 5 carbons; R₁₁, R₁₂, R₁₃ and R₁₄ are independently selected from the group consisting of hydrogen, hydroxyalkyl of 1-5 carbons atoms, hydroxyl and alkyl of 1-5 carbons atoms; X is halogen and Y⁺ is a pharmaceutically acceptable cation.

36. The method of claim 35, wherein Q is Te.

37. The method of claim 36, wherein Y⁺ is NH₄⁺.

38. The method of claim 37, wherein the compound has the formula: 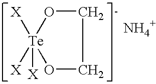
wherein X is halogen.

39. The method of claim 38, wherein the compound is ammonium trichloro(dioxoethylene-O,O′)tellurate (AS101).

40. The method of claim 35 wherein the disorder or disease is selected from insulin resistance; hypertension; dyslipidemia; hyperlipidemia; cardiovascular disease; stroke; gastrointestinal disease; gastrointestinal conditions; osteoarthritis; sleep apnea and respiratory problems; and eating disorders.

41. The method of claim 35 wherein the individual is a human subject.

42. The method of claim 35 wherein the individual is a non-human mammal.

43. The method of claim 35 wherein the pharmaceutical composition is administered orally, parenterally, transdermally, topically or by contacting mucous membranes.

44. The method of claim 43 wherein the pharmaceutical composition is administered orally in unit dosage forms selected from solutions, suspensions, capsules and tablets.

45. The method of claim 43 wherein the pharmaceutical composition is administered via a parenteral route selected from intramuscular, intravenous, intradermal and subcutaneous.

46. The method of claim 43 wherein the pharmaceutical composition is suitable for sustained or controlled release.
